(19) United States
(12) Patent Application Publication
(10) Pub. No.: US 2025/0049580 A1
(43) Pub. Date: Feb. 13, 2025

(54) MODULAR SUPPORT CAGE

(71) Applicant: PR Spine GbR, Mainz (DE)

(72) Inventors: Mathias PIPPAN, Mainz (DE); Michael REITH, Mainz (DE)

(73) Assignee: PR Spine GbR, Mainz (DE)

(21) Appl. No.: 18/717,820

(22) PCT Filed: Dec. 7, 2022
(86) PCT No.: PCT/EP2022/084802

§ 371 Date: Aug. 9, 2024

(30) Foreign Application Priority Data
Dec. 8, 2021 (EP) 21213244.3

(51) Int. Cl. A61F 2/44; A61F 2/30; A61F 2/46

(52) U.S. Cl. CPC A61F 2/4455; A61F 2/4611; A61F 2002/30324; A61F 2002/30505; A61F 2002/30604; A61F 2002/443

(57) ABSTRACT

The invention relates to a modular support cage (10) for supporting vortex elements, comprising a distal support section (20) that has at least two end plates (22a, 22b), which lie opposite each other and which can be moved relative to each other. Each end plate (22a, 22b) has a contact surface (24a, 24b), which is oriented outwards and which is designed to contact the element to be supported, and a bearing region (24a, 24b) that is oriented towards the opposite end plate (22a, 22b) and has a proximal support section (40), said proximal support section having at least two contact surfaces (42a, 42b) oriented outwards and a bearing element (44) that can be joined between the bearing regions (24a, 24b) of the end plates (22a, 22b) and supports same such that the end plates (22a, 22b) are held in a specified position.

### FIELD OF THE INVENTION

The invention relates to a modular support cage for supporting vertebral elements.

### STATE OF THE ART

In the event of damage to the intervertebral disc, so-called vertebral cages are used to support the vertebral elements. These are used to support the spine between the individual vertebral elements in place of the damaged intervertebral disc. In order to ensure the most natural possible position of the individual vertebral elements relative to each other, the lordosis of the spine should be simulated. Since the vertebral elements have a greater spacing at the front than at the rear, such a vertebral cage should also be formed thicker at the front so that the natural spacing of the vertebral elements is maintained. In this case, however, the vertebral cages cannot be inserted from behind through the back, but must be surgically inserted through the abdominal wall. Because the front side of the vertebral cage is formed with a greater spacing and the vertebral elements on the rear side cannot be pushed apart to this spacing, the vertebral cage does not fit through the gap between the adjacent vertebral elements. Alternatively, a smaller vertebral cage can be used for insertion from the back, with which, however, the ideal spacing between the vertebrae and the desired lordosis cannot be achieved.

Such a ventral vertebral cage is described, for example, in U.S. Pat. No. 6,562,074 B2. There is disclosed an adjustable bone fusion implant comprising a first plate having an inner surface with a plurality of spaced-apart first support elements protruding therefrom. Each support element has a plurality of protruding teeth. A second plate has an inner surface with a plurality of spaced-apart second support elements protruding from the second plate. Each second support element has at least one tooth or an adjustment hole formed thereon. A region of the plurality of teeth of each first support element mechanically engages the at least one tooth or an adjustment hole of a corresponding second support element such that the first plate and the second plate may be selectively separated to form a chamber therebetween. A reinforcement element is arranged between the first plate and the second plate such that applying a compressive force between the first plate and the second plate exerts a compression on the reinforcement element.

### DESCRIPTION OF THE INVENTION

The object of the invention is therefore to provide a vertebral cage which can be implanted in a minimally invasive manner from the back of the patient independently of the lordosis. This object is achieved by a modular support cage according to claim 1.

A modular support cage according to the invention for supporting vertebral elements comprises a distal support section that has at least two end plates which lie opposite each other and which can be moved relative to each other, wherein each end plate comprises a contact region which is oriented outwards and which is designed to contact the element to be supported, and respectively a bearing region that is oriented towards an opposite end plate, and a proximal support section having at least two contact regions oriented outwards and a bearing element that can be joined between the bearing regions of the end plates and supports the same such that the end plates are held in a specific position. By dividing it into separate front and rear elements, it is possible to first push the distal support section, which is further away from the user, into its position through the narrow gap between the vertebral elements and only then to position the proximal support section. For this purpose, the end plates of the first support section are pressed apart or otherwise moved away from one another, for example with a tool, so that the contact regions of the end plates have an inclination and position in which the vertebral elements are supported in the natural lordosis. The bearing element is then pushed between the mutually spaced end plates so that they are held in the adjusted position. Therefore, when inserted between the vertebrae, the distal support section does not require more space than the proximal support section, and the modular support cage can be implanted from the back for any lordosis. The bearing element is therefore arranged in the end position between the first contact regions. Further, “distal” means a position oriented away from the user of the support cage and “proximal” means a position closer to the user, wherein the user is the person handling the support cage. Furthermore, “modular” in the sense of the invention means a separate division into a front (distal) and a rear (proximal) part and not a separation of the support sections into top and bottom (even if a separation top/bottom is also provided in the embodiment according to the invention at least for the distal support element in order to be able to adjust the lordosis)

Preferably, the contact regions of the distal and proximal support sections are aligned relative to one another and, in particular, form a common plane. This means that the mutually oriented edges of the first and second support sections abut on each other in a stepless manner and, in particular, also have the same pitch, i.e. have no kink in the surface (kinkless). As a result, uniform support of the vertebral elements is secured and a sufficiently large support surface of the entire element is ensured.

The contact regions and the bearing element of the proximal support section are preferably formed in one piece. This facilitates handling and increases stability and safety when inserting the proximal support section.

Furthermore, at least one of the two end plates can be formed with a tapering thickness, so that the contact region has an inclination, which is in particular extended continuously, but preferably all end plates have such a tapering thickness. Likewise, the tapering thickness also preferably applies to the proximal support section. The desired lordosis is reliably provided through a design with a tapering thickness. A solid design or a design with a trabecular structure can improve the stability of the support sections.

The end plates preferably have an abutment, in particular a housing engagement at their distal end, which serves as an abutment for the bearing element. As a result, the end position of the bearing element can be determined precisely and accidental incorrect positioning can be reliably avoided. A housing engagement additionally secures the end plates against accidental lifting off the bearing element.

The bearing element and the bearing regions preferably have step elements at the interface between the distal and proximal support sections. This avoids lateral pivoting of the support sections in the end position and ensures correct alignment. Such a step section can also be used, in particular, as an abutment for the insertion direction. Furthermore, a second housing engagement can also be formed at the distal end of the contact regions, in which the proximal end of the end plates engages and thus secures the cohesion of the end plate on the bearing element.

The at least two outwardly oriented contact regions of the proximal support section preferably each comprise two spaced-apart surfaces, and the at least two outwardly oriented contact regions of the distal support sections are preferably respectively U-shaped. As a result, it is possible in a simple manner to handle the tools for the two support sections without them interfering with one another.

Another aspect of the invention relates to the anchoring of a bearing element and of a support section, so that the support cage can be used more reliably.

Such a support cage for supporting vertebral elements comprises at least one support section which has at least two support plates which lie opposite each other and which can be moved relative to each other, wherein each support plate comprises a contact region which is oriented outwards and which is designed to contact the element to be supported, and respectively a bearing region that is oriented towards an opposite support plate, and a bearing element which can be joined between the support plates and supports the same such that the support plates are held in a specific position. The bearing element and the support sections of the support cage or of the modular support cage further have mutually complementary latching elements at the distal end region, which latching elements interact together in the end position of the bearing element in such a way that the bearing element is connected to the support sections in a non-displaceable manner.

The latching elements (e.g. spring pin and hole) can, of course, also be used with the preceding embodiment, which has the distal and proximal support sections, so that the bearing element and the support sections of the support cage or of the modular support cage at the distal end region can also have mutually complementary latching elements there, which interact together in the end position of the bearing element in such a way that the bearing element is connected to the support sections in a non-displaceable manner.

The latching element ensures in a simple manner that the bearing element can no longer be moved after the adjustment of the lordosis and the insertion of the bearing element for fixing the support sections.

The latching elements are preferably formed as a spring-driven pin and as a housing. This configuration enables the latching element to be reliably triggered when the end position is reached. The spring-driven pin is preferably formed on the bearing element, and the housing is preferably formed on the bearing regions of the support sections. This ensures that the latching element is well accessible and easily reachable for triggering the latching element.

The end plates of the distal support section may have a tool housing, which is formed in particular as a hole. Such a tool housing facilitates handling when inserting the proximal support section. The hole is preferably circular, as a result of which the alignment of the tool to be inserted is insignificant and introduction into the tool housing is facilitated. However, it can also be oval or polygonal (e.g. triangular, quadrangular, hexagonal), which ensures the alignment of the tool.

### BRIEF DESCRIPTION OF THE FIGURES

FIG. 1 shows an isometric view of the modular support cage in an assembled state;

FIGS. 2a and 2b show isometric views of the modular support cage with auxiliary tools when inserting the proximal support section into the distal support section;

FIG. 3 shows a cross-section through the latching element in the distal support section;

FIGS. 4a and 4b show a longitudinal section through the latching element in the distal support section in the open and latched state; and

FIGS. 5a and 5b show isometric views of the proximal side (back side) of the support sections.

### DESCRIPTION OF THE PREFERRED EMBODIMENTS

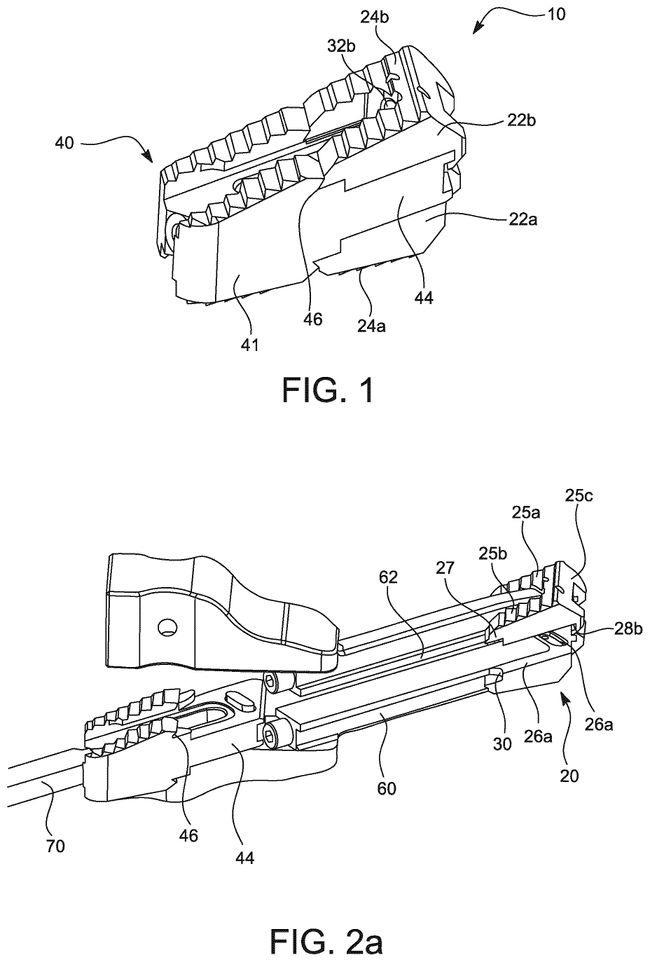

FIG. 1 depicts an assembled modular support cage 10 in an isometric view. Support cage comprises a distal support section 20 and a proximal support section 40.

In the present embodiment, the distal support section 20 is formed in two parts and comprises at least two end plates 22a, 22b. These end plates 22a, 22b each have a contact surface 24a, 24b which is oriented outwards (upwards or downwards in FIG. 1) and which, during use, is in contact with the adjacent vertebral elements. On the side that lies opposite to the contact surfaces 22a, 22b, bearing regions 26a, 26b are provided, which serve for support on a bearing element 44, so that the end plates 22a, 22b are held in the end position by the bearing element. The end plates 22a, 22b are preferably formed in one piece and more preferably U-shaped, wherein the two spaced-apart side arms 25a, 25b extend proximally, i.e. towards the user, and the connecting bridge 25c, which connects the two side arms 25a, 25b, is provided at the distal end of the end plates 22a, 22b. The end plates 22a, 22b are formed with a tapering thickness such that the contact surface 24a, 24b is tilted from the proximal end up to the distal end (at the connecting bridge 25c), wherein the end plates 22a, 22b are thinner at the proximal end and become thicker toward the distal end. However, the inclination of the contact surfaces 24a, 24b can also be achieved by forming only one of the end plates 22a, 22b with a tapering thickness, in which case the tapering should then be formed to be correspondingly larger.

The end plates 22a, 22b preferably have an abutment 30, which can be formed, for example, as a simple step element. Furthermore, at the distal end of the end plates 22a, 22b, a housing engagement 28a, 28b may be provided, which, after engagement with the bearing element 44 described below, firmly holds the end plate 22a, 22b on the bearing element 44. This housing engagement 28a, 28b is preferably also used as an abutment.

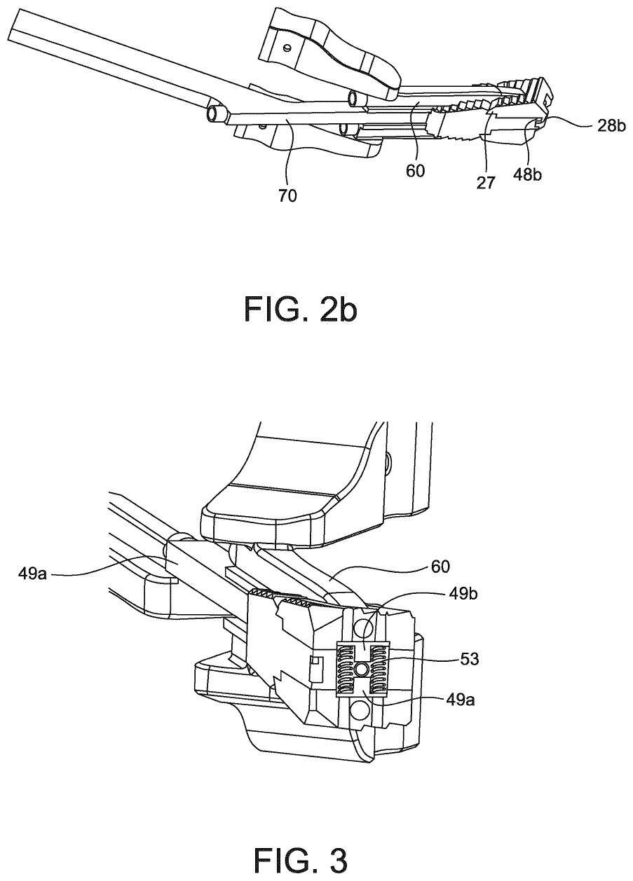

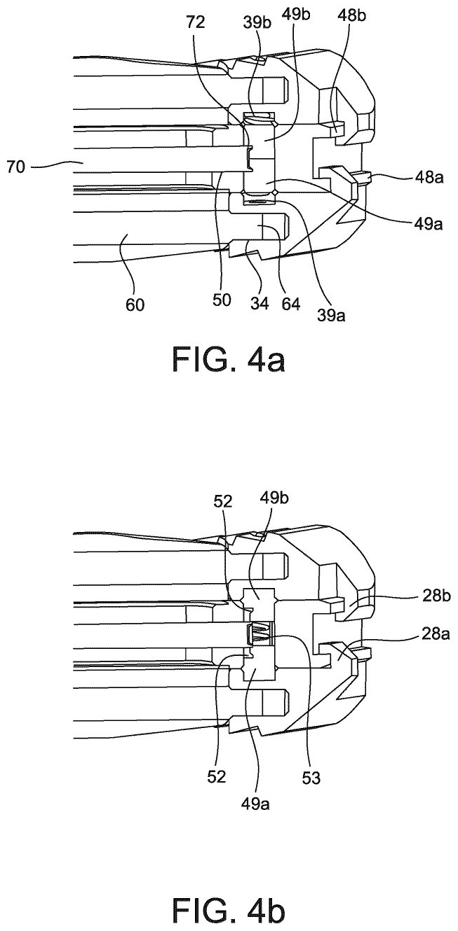

Further, on the inwardly oriented side (opposite the contact surfaces), the end plates have bearing regions 26a, 26b that interact with the bearing element 44. Guide steps, which can be seen in FIG. 2a, are preferably provided on these bearing regions 26a, 26b. These interact with the step sections 62 in the retaining tools 60 and thus guide the retaining tools 60 securely. Furthermore, tool housings 32a, 32b can also be formed on the end plates 22a, 22b, into which the retaining tools 60 can be inserted and which, in particular together with the guide steps, can ensure secure retaining of the end plates 22a, 22b. The tool housing 32a, 32b can in principle be of any desired design, for example oval or polygonal, but a circular hole is however the preferred design of the tool housing.

The proximal support section 40 has a contact section 41 on which the upper and lower contact surfaces 42a, 42b are formed, which during use are in contact with the vertebral elements. Further, the proximal support section 40 comprises a bearing element 44 which extends in the distal direction and which is placed between the end sections 22a, 22b of the distal support section 20 and which contacts the bearing regions 24a, 24b at the top and bottom, respectively. The bearing element 44 is preferably formed in one piece with the contact section 41. Furthermore, it is possible for the bearing element 44 to be formed with a tapering thickness, so that the bearing element 44 becomes thinner towards the distal end. This tapering is then compensated for with a corresponding thickening of the end plates 22a, 22b in order to still obtain the correct lordosis. The tapering serves to support the pushing apart of the end plates 22a, 22b by means of the retaining tool 60 by inserting the bearing element 44.

The proximal support section 40 is preferably U-shaped, so that the contact surfaces 42a, 42b are each provided as two mutually spaced surfaces on a side arm 45a, 45b, and a connecting bridge 45c connects these side arms 45a, 45b at the distal end. In the middle of the connecting bridge 45c, the bearing element 44 extends in the distal direction. At the proximal end of the contact section, a connecting bridge 56 may also be formed, which connects and stabilizes the two side arms 45a, 45b. The connecting bridge 56 further has a recess or a through-hole 54 through which the insertion tool 70 can be guided on the tool housing 50.

Furthermore, the proximal support section 40 can also have a housing engagement 46, which is preferably provided at the distal end of the contact surfaces 44a, 44b, at which the contact surfaces 44a, 44b and the contact surfaces 24a, 24b preferably meet. Here, the housing engagement 46 is formed as a triangular undercut, into which a triangular protrusion 27 of the contact surfaces 24a, 24b of the distal support section 20 is inserted and which is held firmly on the bearing element 44.

For the insertion tool 70, the proximal support section 40 has a tool housing 62 at its proximal end, which in principle can be formed like the tool housing in the distal support section 20 (circular, oval, polygonal). In a preferred embodiment, it is formed as a hole. The tool housing 50 preferably extends as far as a latching mechanism and thus allows the insertion tool 70 to interact with the latching mechanism. In this context, the above-described U-shape is also a great advantage, because it makes it possible, through the spacing between the side arms 45a, 45b, for the tool housing 70 of the connecting bridge 45c to be arranged as close as possible to the latching mechanism described below, and for the latching mechanism to be easier to handle during set-up.

At the distal end of the proximal support section 40, engagement protrusions 48a, 48b are further preferably formed, which interact with the housing engagements 28a, 28b in order to hold both support sections 20, 40 together.

The contact surfaces 22a, 22b, 42a, 42b of the support sections preferably have a pattern. This pattern may comprise, for example, transverse ribs or other reliefs such as simple protrusions. In the embodiment shown, the pattern is represented as triangular (serrated) ribs, which are formed in particular as steps which are adapted to the lordosis, so that the top of the steps is formed almost horizontally and the front sides are formed almost vertically (with an inclination of less than) 10°.

FIGS. 2a and 2b illustrate how the proximal and distal support section are pushed into each other. First, the retaining tools 60 are placed with the step section 62 on the corresponding guide rail of the end plates 22a, 22b and then inserted into the tool housing 34a, 34b. The distal support section is then placed between the vertebrae. The retaining tools 60 shown in the figures may be, for example, the ends of pincers with which the end plates 22a, 22b of the distal support section 20 can be pressed apart. In FIG. 2a, the end plates 22a, 22b are already shown in a mutually spaced position in which the correct lordosis is set. Further, it can be seen how the insertion tool 70 is provided in order to be inserted into the proximal support section 40.

In FIG. 2b, the proximal support section 40 has then been displaced in the direction of the distal support section 20, so that the bearing element 44 is located between the end plates 22a, 22b and contacts their bearing regions 26a, 26b and supports the same. While being pushed together, if present, the engagement protrusions 48a, 48b at the distal end of the support cage have moved into the housing engagements 28a, 28b. Likewise, this has occurred at the junction of the contact surfaces 24a, 24b, 44a, 44b, where the housing engagement 46 has slid over the protrusion 27.

In this position, a latching mechanism can be provided in the modular support cage, which connects the bearing element 44 fixedly and in particular also irreversibly to the end plates 22a, 22b. Such a latching mechanism may also be used in a non-modular support cage (no separation in distal and proximal), which then has two support sections which are formed in one piece from a proximal and a distal support section both at the top and bottom. In addition, there is then a bearing element that is inserted between the end plates of the distal support section and supports the same, analogous to the bearing element 44 that supports the distal support section 20. This embodiment is not shown in the figures, but the latching mechanism functions in the same manner as the latching mechanism for the modular support cage explained below.

The latching mechanism preferably comprises two latching elements, which are preferably designed here as a spring-driven latching block 49a, 49b and as a latching housing 39a, 39b, the term latching block also comprising a configuration as a latching pin or latching ball. In the present design, the spring-driven latching block 49 is arranged in the bearing element 44 and the latching housing 39 in the end plates 22a, 22b. Vice versa, the spring-driven latching block 49 can also be arranged in the end plates 22a, 22b and the latching housing in the bearing element 44. The latching block 49a, 49b is biased with a spring that pushes the latching block toward the end plate 22a, 22b. Here, one or more springs 53 are provided for both latching blocks 49a, 49b, which can simultaneously bias both latching blocks 49a, 49b in the respective opposite direction. In particular, only one spring can actually be provided. The latching blocks 49a, 49b are thus held within the bearing element 44 until the latching housing is reached and are then pressed into the latching housing by the spring 53.

However, so that the latching blocks 49a, 49b do not have to be laboriously held in the bearing element with a tool until the latching blocks 49a, 49b are clamped under the end plates 22a, 22b, locking cavities 52 are also preferably provided in the latching blocks 49a, 49b, which interact with the retaining protrusions 72 of the insertion tool 70. Here, the latching blocks 49a, 49b are compressed from the outside against the spring, and then the retaining protrusions 72 of the insertion tool 70 are first inserted into the tool housing 50 of the proximal support section 40 and then into the locking cavities 52. Because the springs push the latching blocks 49a, 49b outwards, the locking cavities 52 interlock with the retaining protrusions 82 and improve their retention. Only when the proximal support section 40 is actually in the end position, the latching blocks 49a, 49b lie below the corresponding housings 39a, 39b. The insertion tool is then pulled out of the tool housing 50 and the retaining protrusions 72 are thus also removed from the locking cavities 52. As a result, the biased springs press into the latching housings 39a, 39b, and the latching blocks 49a, 49b fix the bearing element 44 in the end plates, so that it is no longer possible to pull out without removing the latching blocks from the latching housings.

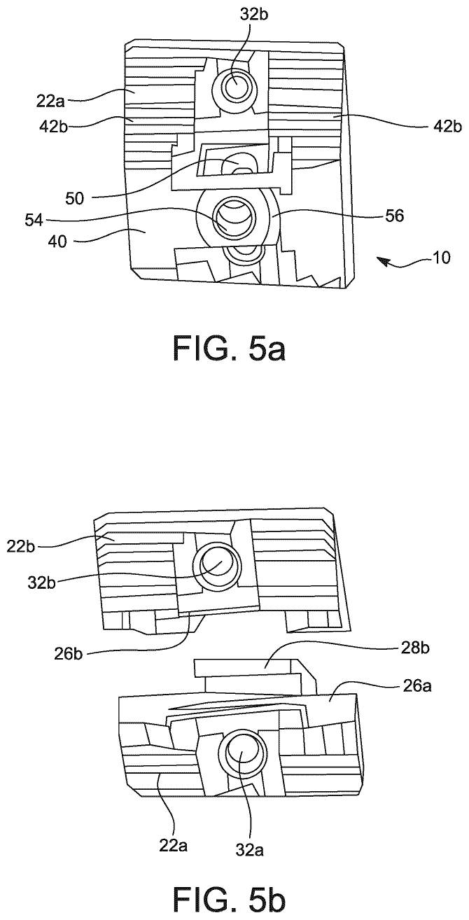

FIG. 5a shows a view of an assembled modular support cage 10. From this view, there are the tool housings 32a, 32b of the distal support section and the tool housing 50 of the proximal support section 40. The tool housing 50 of the proximal support section 40 is provided on the bearing element 44.

Preferably, a further hole 54 or a recess 54 is provided on a proximal reinforcing strut 56, which connects and thus stabilizes the contact surfaces 42a, 42b with the opposite contact surfaces 42a, 42b.

In FIG. 5b, the distal support element 20 is depicted in such a way that the tool housings 32a, 32b can be seen from behind. The housing engagement 28a can also be clearly seen.

### LIST OF REFERENCE SIGNS

Modular support cage 10
Distal support section 20
End plate 22a, 22b
Contact surface 24a, 24b
Side arm 25a, 25b
Connecting bridge 25c
Bearing region 26a, 26b
Protrusion 27
Housing engagement 28a, 28b
Abutment 30
Tool housing 32a, 32b
Latching housing 39a, 39b
Proximal support section 40
Contact section 41
Contact surface 42a, 42b
Bearing element 44
Housing engagement 46
Spring 47a, 47b
Engagement protrusion 48a, 48b
Spring-driven latching block 49a, 49b
Tool housing 50
Locking cavities 52
Spring 53
Through hole 54
Connecting bridge 56
Retaining tool 60
Step section 62
Push-in protrusion 64
Insertion tool 70
Retaining protrusion 72

1. Modular support cage (10) for supporting vertebral elements, comprising:
a distal support section (20) which has at least two support plates (22a, 22b) which lie opposite each other and which can be moved relative to each other, wherein each end plate (22a, 22b) has a contact surface (24a, 24b) which is oriented outwards and which is designed to contact the element to be supported and respectively a bearing region (24a, 24b) that is oriented towards an opposite end plate (22a, 22b); and
a proximal support section (40) which has at least two contact surfaces (42a, 42b) which are oriented outwards and a bearing element (44) that can be joined between the bearing regions (24a, 24b) of the end plates (22a, 22b) and supports the same such that the end plates (22a, 22b) are held in a specific position.

2. Modular support cage (10) according to claim 1, wherein the contact surfaces (24a, 24b, 42a, 42b) of the distal and proximal support sections (20, 40) are aligned relative to each other.

3. Modular support cage (10) according to claim 1, wherein the contact surfaces (42a, 42b) and the bearing element (44) of the proximal support section (40) are formed in one piece.

4. Modular support cage (10) according to claim 1, wherein the at least two end plates (22a, 22b) and/or at least partially the proximal support section (40) are formed with a tapering thickness such that the contact surfaces (24a, 24b, 42a, 42b) are inclined.

5. Modular support cage (10) according to claim 1, wherein the end plates (22a, 22b) have an abutment for the bearing element (44).

6. Modular support cage (10) according to claim 1, wherein the bearing element (44) and the bearing regions (24a, 24b) have step elements (30) at the interface between the distal and proximal support section (20, 40).

7. Modular support cage (10) according to claim 1, wherein the at least two outwardly oriented contact regions of the proximal support section have respectively two spaced-apart surfaces, and the at least two outwardly oriented contact regions of the distal support sections are respectively U-shaped.

8. Support cage for supporting vertebral elements comprising:
at least a support section which has at least two support plates which lie opposite each other and which can be moved relative to each other, wherein each support plate comprises a contact region which is oriented outwards and which is designed to contact the element to be supported, and respectively a bearing region that is oriented towards an opposite support plate; and
a bearing element that can be joined between the support plates and supports the same such that the support plates are held in a specific position,
OR
modular support device (10) according to claim 1,
characterised in that
the bearing element (44) and the support plates of the support cage or the end plates (22a, 22b) of the modular support cage (10) further have mutually complementary latching elements (39a, 39b, 49a, 49b) at a distal end region, which interact together in the end position of the bearing element (44) in such a way that the bearing element (44) is connected to the support sections or end plates (22a, 22b) in a non-displaceable manner.

9. Support cage or modular support cage (10) according to claim 8, wherein the latching elements are formed as a spring-driven latching block (49a, 49b) and as a latching housing (39a, 39b).

10. Support cage or modular support cage (10) according to claim 9, wherein the spring-driven latching block (49a, 49b) is formed on the bearing element (44) and the latching housing (39a, 39b) on the bearing regions (26a, 26b) of the end plates (22a, 22b).

11. Support cage or modular support cage (10) according to claim 1, wherein the end plates of the distal support section and/or the proximal support section have a tool housing (34a, 34b, 50).
